(19) United States
(12) Patent Application Publication
(10) Pub. No.: US 2025/0306007 A1
(43) Pub. Date: Oct. 2, 2025

(54) DETECTING APOPTOTIC BODIES BY IMPEDANCE CYTOMETRY AS AN INDICATOR OF DRUG SENSITIVITY

(71) Applicants: Nathan SWAMI, Charlottesville, VA (US); Todd BAUER, Charlottesville, VA (US); Armita SALAHI, Charlottesville, VA (US); Carlos Manuel Fernandes HONRADO, Macedo de Cavaleiros, Braganca (PT); University of Virginia Patent Foundation, Charlottesville, VA (US)

(72) Inventors: Nathan Swami, Charlottesville, VA (US); Todd Bauer, Charlottesville, VA (US); Armita Salahi, Charlottesville, VA (US); Carlos Manuel Fernandes Honrado, Macedo de Cavaleiros, Braganca (PT)

(21) Appl. No.: 18/866,431

(22) PCT Filed: May 17, 2023
(86) PCT No.: PCT/US2023/067113

§ 371 Date: Nov. 15, 2024

Related U.S. Application Data
(60) Provisional application No. 63/342,741, filed on May 17, 2022.

(51) Int. Cl. G01N 33/50; G01N 15/10; G01N 15/1031; G01N 33/574

(52) U.S. Cl. CPC G01N 33/5011; G01N 15/1031; G01N 33/57492; G01N 2015/1006; G01N 2015/1029

(57) ABSTRACT

A microfluidic system can be used to quantify apoptotic bodies (ABs) with single-cell sensitivity, providing real-time information regarding the presence, and properties of ABs. Different subpopulations of ABs can thus be distinguished from one another to quantify cellular dis-assembly and drug sensitivity of the cancer cells under test. Impedance measurement can be performed by flowing secreted bodies at a substantially single-particle sensitivity. A plurality of electrical impedance magnitude and phase parameters of the biological sample can be measured within the flow cell structure, corresponding to a specified range of frequencies to help determine a biological characteristic of the cancer cells.

### CLAIM OF PRIORITY

This application claims priority to U.S. Provisional Application Ser. No. 63/342,541, filed on May 17, 2022, which is incorporated by reference herein in its entirety, and the benefit of priority of which is claimed herein.

### STATEMENT REGARDING FEDERALLY SPONSORED RESEARCH OR DEVELOPMENT

This invention was made with government support under TR003015 awarded by the National Institutes of Health and FA2386-18-1-4100 and W911NF-17-3-003 awarded by Department of Defense. The government has certain rights in the invention.

### BACKGROUND

Impedance-based cytometry can be used such as to measure electrical properties of cells, sub-cellular bodies, and cellular aggregates. In single-cell impedance cytometry, the detection region can include or use pairs of parallel-facing electrodes, fabricated within a channel. An AC signal can be applied to the first electrodes; and the difference in current flowing through the channel is acquired by the second electrodes and measured by detection circuitry. The impedance changes caused by the presence of a particle between the electrode pair are then translated into a change in the current signal being measured, as the current path becomes disturbed. When a particle passes the center of the detection region, individual particle signals are generated. Individual particle signals can be retrieved by signal processing circuitry and, subsequently, are used to plot population distribution and perform data analysis.

### SUMMARY

Biophysical cellular information obtained at single-cell sensitivity can be used within analytical and separation platforms to help associate cell phenotype with markers of disease, infection, and immunity. Certain frequency-modulated, electrically driven microfluidic measurement and separation systems can help enable identification of single cells, e.g., based on biophysical information. Such identification can be based on detected biophysical information including, e.g., a cellular size, shape, subcellular membrane morphology, and cytoplasmic organization.

For example, cancer cell cultures can be analyzed for presence of sub-cellular apoptotic bodies (ABs) to help quantify cellular states, e.g., a live state, an apoptotic state, or a necrotic state. These ABs generally coincide with vesicles or micro-vesicles secreted into a culture media during a drug treatment and the vesicles can serve as identifiers for drug sensitivity. Identification and stratification of these ABs to quantify cell dis-assembly using certain approaches can be present challenges due to the compositional diversity of the ABs. For example, ABs can include a complex mixture of proteins, lipids, and nucleic acids that can be present in a variety of forms and sizes. As a result, such ABs can be difficult to differentiate from other cellular matter, such as cellular debris or exosomes.

Certain approaches to detect and quantify apoptotic bodies, e.g., flow cytometry and fluorescence imaging, can be limited to identifying relatively few classes of biological markers. Such approaches can also be limited in terms of the overall sensitivity and specificity, as well as limited in the ability to obtain sufficient information from a single sample. For example, fluorescence imaging can require labeling of the target material with a fluorescent probe, which can reduce the overall sensitivity of the imaging and can limit the ability to identify certain types of biological markers. The present inventors have recognized a clinical need for an alternative approach to better detect and quantify ABs and improve time-sensitive assessment of a viability or cellular resistance to a chemotherapeutic agent.

To help address these challenges, certain frequency-modulated, electrically driven microfluidic measurement and separation techniques can be used to detect and quantify ABs. These systems can allow for single-cell sensitivity and can provide real-time information regarding the presence, size, and composition of the apoptotic bodies. Additionally, these systems can enable identification of apoptotic bodies based on their electrical properties, such as impedance, which can be used to differentiate between ABs and other cellular matter and to help distinguish subpopulations of vesicles corresponding to ABs from one another. This information can be used to help quantify cellular dis-assembly and drug sensitivity of the cancer cell culture under test.

This document details an approach involving microfluidic measurement or separation of supernatants in the media of gemcitabine-treated pancreatic tumor cultures to detect biophysical information of the ABs. In an example, such ABs can exhibit phenotypic resemblance to lifted apoptotic cells and enables shape-based stratification within distinct size ranges.

Detected biophysical information indicating a presence of ABs can be useful in predicting drug response and toxicity using in vitro mammalian tumor cells. For example, a machine-implemented method for predicting drug response and toxicity using such tumor cells can include receiving a supernatant biological sample of secreted bodies in a flow cell structure that can be obtained from in vitro mammalian tumor cells concurrent with treatment with a specified chemotherapeutic agent. For example, the received in vitro mammalian tumor cells can correspond to a specified patient-derived tumor. An alternating current (AC) electrical stimulus can be generated and delivered to a set of electrode structures that can be electrically coupled with the flow cell structure for impedance measurement of flowing secreted bodies, e.g., at a substantially single-particle sensitivity. In response to the electrical stimulus, a plurality of electrical impedance magnitude and phase parameters of the biological sample can be measured within the flow cell structure, e.g., corresponding to a specified range of frequencies.

The measured electrical impedance parameters of the biological sample can then be compared with respective electrical impedance parameters corresponding to model live floating cells, model apoptotic cells, model apoptotic bodies, micro-vesicles corresponding with apoptotic cells, or a combination thereof. For example, a measured impedance phase versus size distribution of the biological sample can be compared with impedance phase versus size distribution of the model apoptotic cells and live floating cells. Based on these compared parameters, a biological characteristic corresponding to a chemotherapeutic drug sensitivity of the in vitro mammalian tumor cells in the biological sample can be determined. For example, the biological characteristic can correspond with live, apoptotic, or necrotic states of the in vitro mammalian tumor cells in the biological sample. The biological characteristic can also indicate a presence of apoptotic bodies (ABs) and micro-vesicles in the biological sample.

In an example, one or more impedance characteristics of a target vesicle or micro-vesicle can be altered, e.g., via synthetic nanostructures configured to specifically bind with surface proteins on the vesicles or micro-vesicles. For example, the synthetic nanostructures can alter impedance characteristics based on a protein type or expression level, e.g., to help stratify different vesicle subpopulations. A specific subpopulation of vesicles from the biological sample can also be isolated or separated, e.g., based on their characteristic impedance characteristics. One or more distinguished subpopulations of vesicles can be used such as to determine or predict a drug resistance of the in vitro mammalian tumor cells to the specified chemotherapeutic agent. Here, the distinguished subpopulations of vesicles corresponding to the ABs can be compared with a tumor microenvironment (TME) model. Ultimately, detected biological characteristics of the in vitro mammalian tumor cells can factored in establishing or adjusting a drug treatment plan for a patient corresponding to the in vitro mammalian tumor cells.

This document also describes a microfluidic system for predicting drug response and toxicity using in vitro mammalian tumor cells. Such a microfluidic system can include or use a main channel defining an inlet and an outlet. The main channel can be sized and shaped such as to receive a supernatant biological sample of secreted bodies obtained from in vitro mammalian tumor tissue concurrent with treatment with a specified chemotherapeutic agent, at the inlet. The microfluidic system can also include a set of electrode structures that can be coupled with the main channel for impedance measurement of flowing secreted bodies at a substantially single-particle sensitivity. The microfluidic system can also include a waveform generator for delivering an AC electrical stimulus to the set of electrode structures.

The microfluidic system can include or be communicatively coupled to a processor. The processor can include or can be communicatively coupled to measurement circuitry to measure, via the electrodes in response to the electrical stimulus, a plurality of electrical impedance magnitude and phase parameters of the biological sample within the flow cell structure, corresponding to a specified range of frequencies, e.g., generated by the waveform generator. For example, the specified range of frequencies can include frequencies within a range of 500 kilohertz (kHz) to 50 megahertz. The processor can compare the measured electrical impedance parameters of the biological sample with respective electrical impedance parameters corresponding to at least one of model live floating cells, model apoptotic cells, model apoptotic bodies, or micro-vesicles corresponding with apoptotic cells. In an example, the processor can also determine, based on the compared parameters, a biological characteristic corresponding to a chemotherapeutic drug sensitivity of the in vitro mammalian tumor cells in the biological sample. In an example, the microfluidic system can also include at least one focusing flow channel feeding the main channel to establish or adjust a flow rate of the biological sample. For example, the focusing flow channel can establish the flow rate at a throughput within a range of 200-500 particles per second.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A shows an example of a microfluidic system for predicting drug response and toxicity using in vitro mammalian tumor cells.

FIG. 1B is a schematic example of a progression of drug resistance onset of in vitro cancer cells.

FIG. 1C is a schematic example of a technique for utilizing interaction of cancer cells and CAFs to help further create phenotypes of in vitro cancer cells.

FIG. 2A depicts examples of two dimensional (2D) density plots of impedance metrics for cancer cell samples exposed to a chemotherapeutic drug at varying drug concentrations.

FIG. 2B depicts examples of 2D density plots of impedance metrics for cancer cell samples exposed to a chemotherapeutic drug at varying drug concentrations.

FIG. 2C depicts examples of three dimensional (3D) density plots of impedance metrics for untreated cancer cell samples.

FIG. 2D depict examples of 3D density plots of impedance metrics for cancer cell samples exposed to a chemotherapeutic drug.

FIG. 2E depicts plots showing impedance metrics for various ABs for treated and untreated cancer cell cultures.

FIG. 2F depicts plots showing impedance metrics for various ABs for treated and untreated cancer cell cultures.

FIG. 3A is a schematic showing modification of a vesicle using receptor-bound nanostructures to help distinguish impedance metrics during impedance cytometry.

FIG. 3B depicts a microfluidic device micro-sampling and microfluidic separation of secreted bodies.

FIG. 4A is a plot of impedance metrics of live floating cancer cells, secreted from chemotherapeutic drug-treated cancer cell cultures, as compared with phenotypes of the drug-resistant subpopulation of live adherent cells lifted a cancer cell culture.

FIG. 4B is a plot of impedance metrics of live floating cancer cells, secreted from chemotherapeutic drug-treated cancer cell cultures, as compared with phenotypes of the drug-resistant subpopulation of live adherent cells lifted a cancer cell culture.

FIG. 4C is a plot of impedance metrics of live floating cancer cells, secreted from chemotherapeutic drug-treated cancer cell cultures, as compared with phenotypes of the drug-resistant subpopulation of live adherent cells lifted a cancer cell culture.

FIG. 4D is a plot of impedance metrics of live floating cancer cells, secreted from chemotherapeutic drug-treated cancer cell cultures, as compared with phenotypes of the drug-resistant subpopulation of live adherent cells lifted a cancer cell culture.

FIG. 5 is a flowchart that describes a machine-implemented method for predicting drug response and toxicity.

FIG. 6 is a block diagram illustrating components of a machine.

### DETAILED DESCRIPTION

Apoptosis or programmed cell death can provide certain biological cues to enable cell clearance by phagocytes and mediate their communication within the broader cellular micro-environment. Cellular apoptosis can involve a formation of plasma-membrane blebs on the cell surface, thin membrane protrusions, microtubule spikes, apoptopodia and beaded structures on the plasma membrane, and eventual cell fragmentation and distribution of cellular material into sub-cellular bodies and vesicles (also referred to herein as micro-vesicles). As described herein, the term apoptotic bodies (ABs) can include sub-cellular (e.g., about 1 μm to about 5 μm in diameter), membrane-bound apoptotic extracellular vesicles, with differing size, shape, and internal composition. The kinetics of cell disassembly at each apoptotic stage and their stratification can provide valuable information on the efficacy of drug treatments or the emergence of drug resistance in cancer cells. Cancer cell cultures can be evaluated for the presence of these subcellular ABs to gauge cellular states, e.g., a live state, an apoptotic state, or a necrotic state. However, the diversity of ABs in terms of proteins, lipids and nucleic acids can make it difficult to differentiate them from other cellular matter, such as exosomes and cellular debris.

Certain approaches to evaluating cancer cell cultures for ABs, such as performing proliferation assays in cell cultures or histology of tissue samples can be insufficient in providing requisite quantitative information on sub-cellular ABs. Also, approaches involving microscopy of adherent cells can be limited to imaging relatively few AB numbers that are not statistically relevant. Certain flow cytometry-based approaches can be used to help achieve measurement of statistically relevant numbers of ABs and cells under apoptotic conditions. A problem, however, with these flow cytometry-based approaches is that it can be challenging to identify the appropriate fluorescence stain for each AB type and to minimize dependence of the measurements on differential dye penetration kinetics across each AB type. This is due to the fact that ABs have distinct sub-cellular contents that are tailored for specific functions in the microenvironment. Other label-free approaches to analyze sub-cellular ABs, e.g., Raman spectroscopy, fluorescence lifetime measurements, optical tomography, or conductivity and dielectrophoresis measurements can lack the desired throughput and sensitivity for measuring the large event numbers at single-particle sensitivity, as needed for statistically relevant stratification of sub-cellular ABs.

Chemotherapeutic agents generally exhibit a high degree of patient-to-patient variability in drug sensitivity and cytotoxicity. For example, since these drugs can broadly interfere with cell replication or DNA repair pathways, rather than being targeted to interfere with particular cell receptors or proteins, their action cannot be predicted by genetic and transcriptional markers. Given the relatively short timeframe available to certain cancer patients (e.g., pancreatic cancer), who can have a median survival duration between about 3-7 months, there is a need for in vitro cell-based assays to help screen pre-clinical drug targets to gauge clinical benefit by using patient derived tumor materials.

The present inventors have recognized a need for an alternative approach to detect and quantify apoptotic bodies (ABs) to help improve time-sensitive assessment of cellular resistance or viability to a chemotherapeutic agent. To this end, frequency-modulated, electrically driven microfluidic measurement and separation systems can be used to detect and quantify ABs with single-cell sensitivity, and to provide real-time information regarding the presence, size, composition, and electrical properties of ABs. This information can be used to help differentiate between ABs and other cellular material, and to distinguish between different subpopulations of ABs from one another, in order to help quantify cellular dis-assembly and drug sensitivity of a cancer cell culture under test. In an example, the techniques described herein can be used to assist monitoring of a cancer cell culture by live cell imaging, e.g., to complement live cell imaging of the adhered culture with cytometry of secreted bodies in the culture supernatant, including floating cancer cells, apoptotic bodies, micro-vesicles, and exosomes to provide additional information on the onset of drug resistance and metastasis. In this manner, drug-induced transformations within adhered cell cultures can be monitored without lifting the cells, thereby providing temporal information without disturbing the culture. In an example, high frequency impedance phase versus size distribution of ABs determined by impedance cytometry of supernatants in the media of tumor cultures treated with a chemotherapeutic agent. Such tumor cultures can exhibit phenotypic resemblances to lifted apoptotic cells and can be used, e.g., with dielectric shell models for size and shape-based stratification. This can include AB subpopulations of small spherical vesicles of <2.6 μm that can exhibit low impedance phase (<0.3), mid-sized oblate ABs (3-8 μm) that can exhibit high impedance phase (>0.5) and wider sized ABs (3-14 μm) that can exhibit low impedance phase (<0.3) that can arise from spherical or prolate ABs. Stratification of ABs can be especially important in assessing cellular resistance or viability of the chemotherapy agent, since variations in drug sensitivity kinetics and drug resistance of certain tumors can be associated with the presence of particular AB types and their relative proportions.

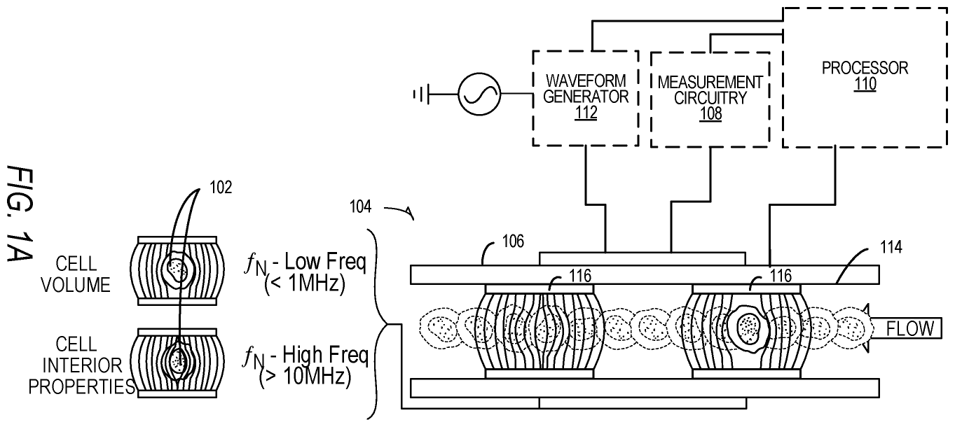

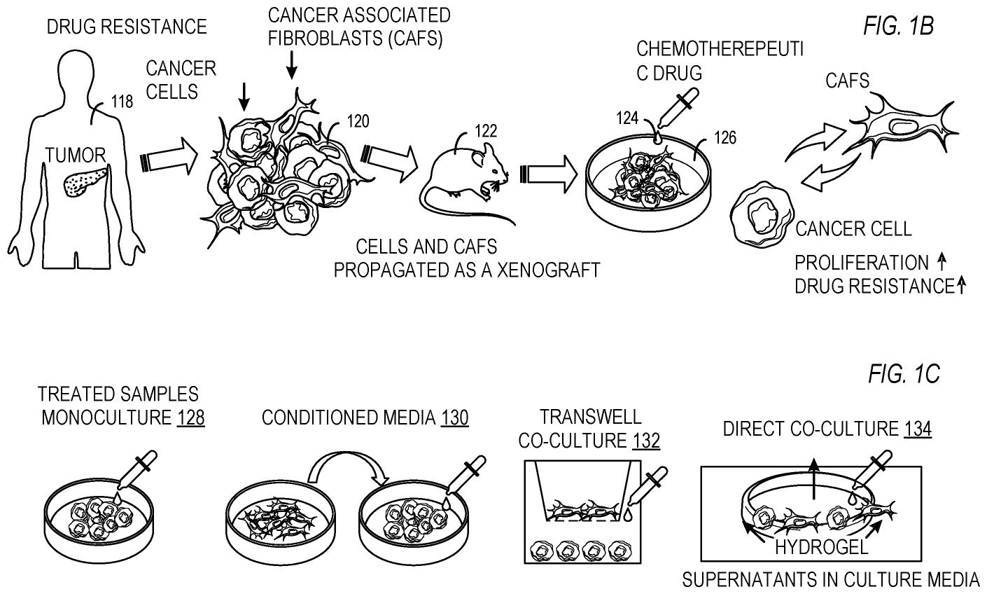

FIG. 1A shows an example of a microfluidic system 100 for predicting drug response and toxicity using in vitro mammalian tumor cells. The microfluidic system 100 can be configured to receive a biological sample or culture 102 within a test cell 106 of an impedance cytometry device 104. As depicted in FIG. 1A, the microfluidic system 100 can be used to classify a test cell 106 based on biophysical information, such as impedance metrics. The microfluidic system 100 can be used such as to receive biological sample or culture 102 and identify a supernatant biological sample of secreted bodies obtained from in vitro mammalian cancer cells, e.g., corresponding with or treated by a specified chemotherapeutic agent or a specified patient-derived tumor being present in the biological sample or culture 102.

In an example, the test cell 106 can include a main channel 114 defining an inlet and an outlet, the main channel 114 included such as to receive the biological sample or culture 102, at the inlet. The biological sample or culture 102 can be passed through the main channel 114 of the test cell 106, where the impedance metrics of the cancer cells can be measured. For example, the microfluidic system 100 can include a set of electrode structures 116 that can be coupled with the main channel 114 for impedance measurement of flowing secreted bodies at a substantially single-particle sensitivity, a waveform generator 112 included such as to deliver an AC electrical stimulus to a set of electrode structures 116.

The impedance cytometry device 104 can include measurement circuitry 108, such as electrodes, amplifiers, and other components, to measure impedance metrics associated with the test cell 106. The impedance metrics can include, for example, single-cell capacitance and/or other frequency-dependent impedance metrics, such as an impedance phase angle or an impedance magnitude. The measurement circuitry can determine or receive measurements of electrical impedance data of the specimen using a specified range of frequencies, and electrical impedance parameters can be extracted from the electrical impedance data. The electrical impedance parameters can correspond to or characterize biophysical or electrophysiologic features of the specimen. In an example, the impedance parameters can correspond to one or more of electrical size value, cell volume, impedance phase value, impedance magnitude value, or capacitance of constituents comprising the biological specimen.

Individual cells from the biological sample or culture 102 can flow (e.g., propelled via a focusing flow channel of the impedance cytometry device 104) through the main channel 114 of the test cell 106 at a specified throughput (e.g., within a range of about 200 cells/s to about 500 cells/s) past electrodes or microelectrodes under an AC electric field applied over a specified range of frequencies (e.g., within a range between about 0.5 megahertz (MHz) to 50 MHz). In an example, an impedance of respective detected specimen can be measured by the measurement circuitry 108 concurrently or simultaneously using a plurality of discrete frequencies, e.g., a reference frequency within a range of about 15 MHz and about 20 MHz, and one or more analysis frequencies within a specified analysis frequency range. The reference frequency can be used such as to gate reference particles versus cells or to account for temporal variations within the impedance cytometry device 104. As depicted in FIG. 1A, a plurality of specified analysis frequency ranges can be used in the microfluidic system 100, such corresponding to respective constituents of the biological specimen. In an example, analysis frequencies less than 1 MHz can be used to measure electrical impedance parameters corresponding to a cell volume.

Analysis frequencies within a range of about 1 MHz to about 10 MHz can correspond to electrical impedance parameters corresponding to a cellular membrane property. Analysis frequencies greater than about 10 MHz can correspond to electrical impedance parameters corresponding to cellular interior properties such an electrophysiology of a nucleus or organelle contained within the specimen. Analysis frequency ranges can be, e.g., from DC or near-DC to about 1 MHz, within a range of about 1 MHz to about 10 MHz, or at a frequency greater than about 10 MHz.

The impedance cytometry device 104 can also include processor 110, such as a microprocessor, microcontroller, or other system, to analyze the impedance metrics or generate a classification result for the test cell 106. The processor 110 can receive the measured impedance metrics associated with the individual cells from the biological sample 102, and can compare the impedance metrics to the impedance metrics associated with a model cell variety, e.g., at least one of at least one of model live floating cells, model apoptotic cells, model apoptotic bodies, or micro-vesicles corresponding with apoptotic cells. In an example, the processor 110 can determine, based on the compared parameters, a biological characteristic corresponding to a chemotherapeutic drug sensitivity of the in vitro mammalian cancer cells in the biological sample. The processor 110 can determine a biological characteristic corresponding with at least one of live, apoptotic, or necrotic states of the in vitro mammalian cancer cells in the biological sample. For example, the processor 110 can compare a measured impedance phase versus size distribution of the biological sample with impedance phase versus size distribution of the model apoptotic cells and live floating cells. Here, the processor 110 can distinguish subpopulations of modified vesicles corresponding to the ABs based on the measured impedance phase and size distribution of the biological sample.

In an example, the processor 110 can be included such as to compare the distinguished subpopulations of vesicles corresponding to the ABs with a tumor microenvironment (TME) model. For example, the TME model can correspond to a model of a tissue microenvironment of a cancer cell or a model of a tissue microenvironment of a tumor tissue. In an example, the TME model can include at least one of a tumor microenvironment component, an immunotherapy component, a chemotherapeutic drug component, or a radiation therapy component of the TME model. In an example, the processor 110 can receive a user input associated with conditions or parameters of the in vitro mammalian cancer cells in the biological sample.

The processor 110 can also generate instructions for to assist in establishing or adjusting a drug treatment plan based on the determined at least one distinguishable biophysical feature of the biological sample, such as a presence of apoptotic bodies (ABs) and micro-vesicles in the biological sample. For example, the processor 110 can cause the display device or output device to provide a visual representation of at least one of the determined biological characteristics or a risk associated with a chemotherapeutic drug treatment response. In an example, the processor 110 can cause the display device or output device to provide a visual representation of a sensitivity of the in vitro mammalian cancer cells to a chemotherapeutic drug or a radiation therapy.

FIG. 1B is a schematic example of a progression of drug resistance onset of in vitro cancer cells. In an example, a tumor tissue sample 120 can be collected from a cancer patient 118 such as via surgical resection or biopsy. Cells from the tumor tissue sample 120 can be propagated, e.g., within immunocompromised mice as a xenograft 122. For example, cells from the tumor tissue sample 120 can exhibit cancer associated fibroblasts (CAFs), a heterogeneous population of cells associated with tumor progression and metastasis. Once propagated, the line established from the xenograft 122 can be maintained in culture for treatment from a chemotherapeutic drug 124, such as gemcitabine, a fluorouracil, doxorubicin, or any other drug used in the treatment of cancer. In an example involving gemcitabine treatment on highly tumorigenic cells derived from the xenograft 122, the range of gemcitabine levels that lead to drug sensitivity can be assessed by cell proliferation assays 126. Depending on the drug treatment, the in vitro cancer cells can be drug resistant, e.g., with varying degrees of resistance to one or more drugs. The CAFs can also be further propagated in vitro and subjected to additional drug treatment(s) to further examine the drug resistance of the cancer cells.

In an example, the cell proliferation assays 126 can be used to compare phenotypes over the measured chemotherapeutic drug 124 concentration ranges, the phenotypes including the generation of blebs of varying size on the plasma membrane (at about 0.01 μg mL⁻¹), the presence of large protrusions (at about 0.1 μg mL⁻¹) and the emergence of beaded structures (at about 1 μg mL⁻¹). These structures can be associated with apoptosis-induced cell disassembly, which generates ABs of particular composition and structure. These phenotypes can be used as a model to help identify and stratify ABs in subsequent patient-derived tumor cells using impedance cytometry techniques based on comparison of their single-particle sensitivity levels.

FIG. 1C is a schematic example of a technique for utilizing interaction of cancer cells and CAFs to help further create phenotypes of in vitro cancer cells. For example, a cell proliferation assay 126 can be altered such as to create a plurality adhered cultures of increasing drug resistance. As depicted in FIG. 1C, a cell proliferation assay 126 can be affected by various degrees in increase drug resistance thereof: e.g., (i) monoculture 128, (ii) conditioned media 130, (iii) membrane (e.g., a Transwell™ membrane) co-culture 132, and (iv) Direct 3D co-culture in hydrogel. For example, the monoculture 128 can be used to provide a benchmark for the cell proliferation assay 126, e.g., by providing a standard for the drug resistance phenotype. The conditioned media 130 can be used to provide a measure of the CAF secretome in affecting the drug resistance phenotype of the cell proliferation assay 126. The Transwell™ membrane co-culture 132 can be used to examine the effect of CAF proximity on the drug resistance phenotype of the cell proliferation assay 126. The Direct 3D co-culture in hydrogel 134 can be used to further examine the effect of CAF proximity on the drug resistance phenotype of the cell proliferation assay 126 without the use of a Transwell™ membrane. In an example, lowered drug sensitivity can exhibit more drug resistant cancer cells when subject to interaction with CAFs, especially with Transwell™ and direct co-culture systems.

A creation of drug resistance through interaction of cancer cells with cancer associated fibroblasts (CAFs) can lead to higher viability proportions after treatment with the chemotherapeutic drug 124, which can cause a less sharp increase in the number of floating cancer cells and the number of secreted apoptotic bodies (ABs) than observed with monocultures 128. Live floating cancer cells secreted from adhered chemotherapeutic drug-treated cultures show similarity in various phenotypes to the drug-resistant subpopulation of live adherent cells. Hence, floating cancer cells and secreted ABs in the media provide key information on the adhered culture. For example, the floating cancer cells and secreted ABs can be used to characterize the drug resistance phenotype of the cancer cells, e.g., via a comparison of the single-particle sensitivity levels of the floating cancer cells or secreted ABs.

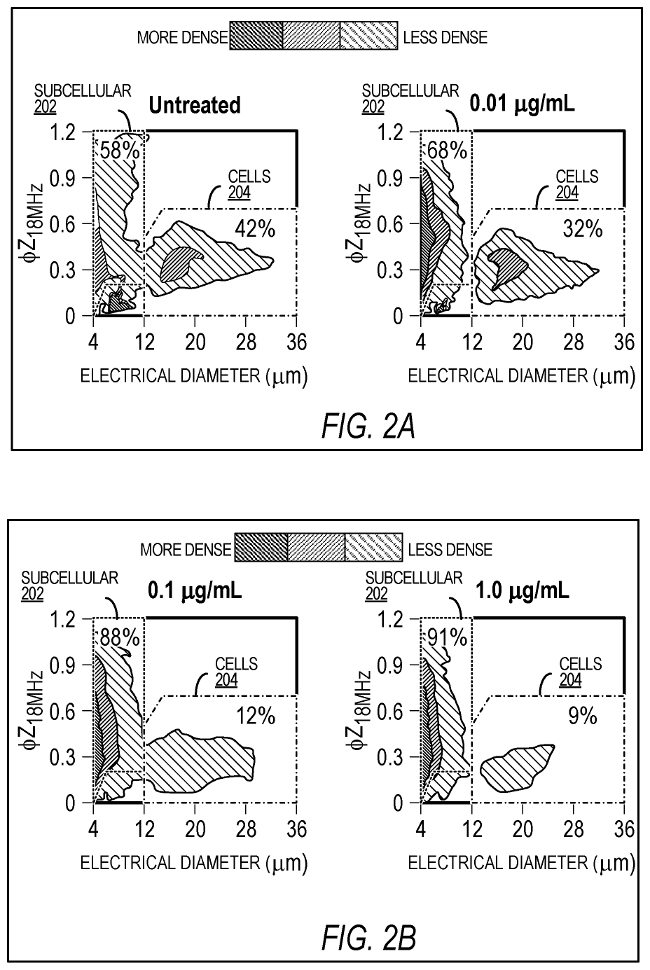

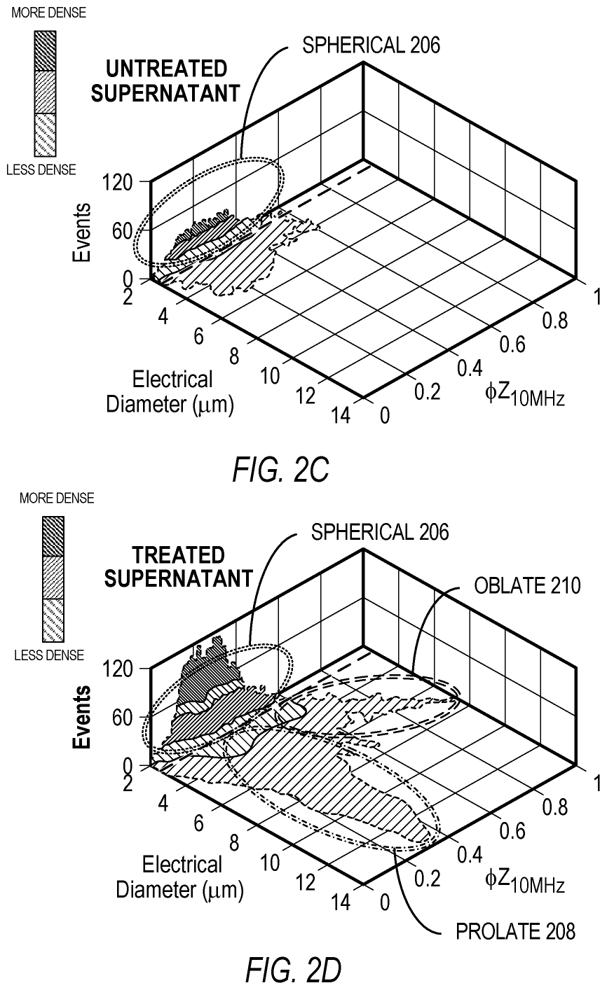

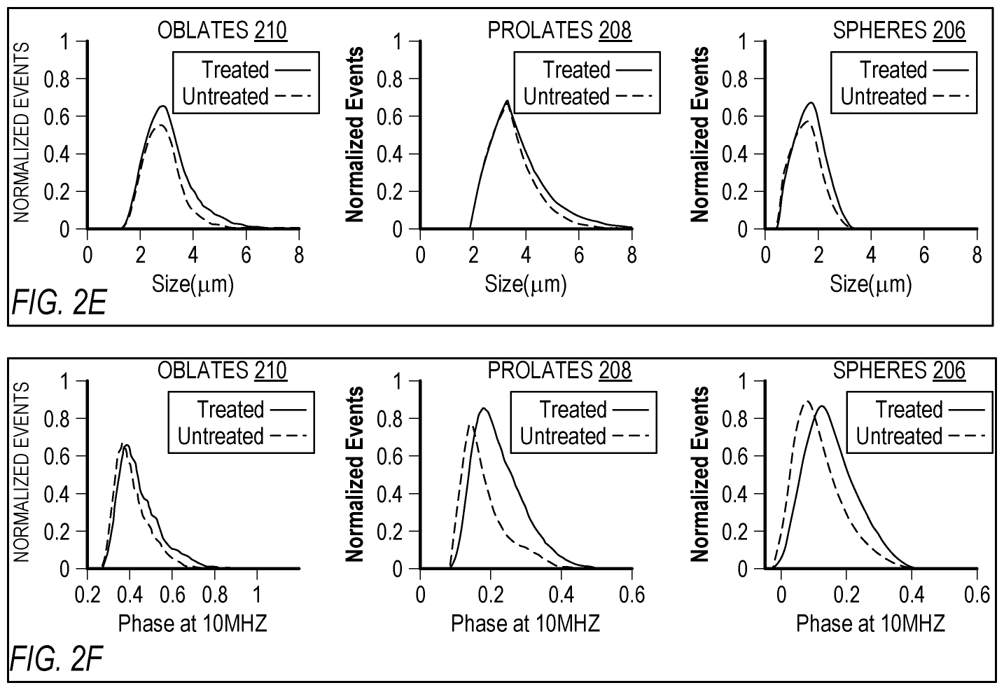

FIG. 2A and FIG. 2B depict examples of two dimensional (2D) density plots of impedance metrics for cancer cell samples exposed to a chemotherapeutic drug at varying drug concentrations. Herein, the “density” and indicated in the provided legend represented on the plots represents a density of events or occurrences at or near a particular 2D or 3D coordinate on the plot. For example, the impedance metrics can include electrical diameter (e.g., \sqrt{3}(|Z|_{0.5 MHz})) versus impedance phase at 18 MHz (e.g., ϕZ_{18 MHz}) for cancer cell samples exposed to a chemotherapeutic drug at varying drug concentrations in a drug treatment period (e.g., about 48 hours). For example, FIG. 2A depicts respective density plots for an untreated cell sample and for a sample treated at a concentration of about 0.01 μg mL⁻¹ of a chemotherapeutic agent (e.g., gemcitabine). FIG. 2B depicts respective density plots for a sample treated at a concentration of about 0.1 μg mL⁻¹ of the chemotherapeutic agent and for a sample treated at a concentration of about 1 μg mL⁻¹ of the chemotherapeutic agent. The electrophysiology of the cancer cells lifted from the culture after various gemcitabine treatment conditions can be measured by impedance cytometry to determine impedance magnitude (|Z|) and impedance phase (ϕZ) at a low (0.5 MHz) and a high frequency (18 MHz) for analyzing individual cellular 204 and/or sub-cellular 202 events. The lipid cell membrane screens the field at low frequencies in high conductivity media to cause insulator-like behavior, which can be used to estimate their electrical diameter from |Z| at 0.5 MHz. At increasing frequencies, capacitive coupling across the cell membrane can cause the cancer cells to present a lower impedance. At a specified threshold frequency (e.g., at about 18 MHz), the impedance signal can be effectively determined by the dielectric properties of the cell interior, rather than properties the cell exterior. This can enable the measurement of each individual cancer cell based on its size (using |Z|_{0.5 MHz}) and its interior electrophysiology (using ϕZ_{18 MHz}) to analyze the phenotypes in a label-free manner.

As depicted in FIG. 2A and FIG. 2B, the rise in sub-cellular events 202 with increasing chemotherapeutic drug concentration can be attributed to release of ABs under drug-induced apoptosis that corresponds with lowering of cell viability and cell proliferation. These sub-cellular events 202 arise from at least one of cell debris and ABs, including micro-vesicle ABs, clusters of ABs of varying size and morphologies, larger apoptotic bodies (>5 μm in diameter) generated later during cell disassembly, or a combination thereof. For example, based on electrical diameter evolution of the gated sub-cellular particles, there is an increase in the size range of particles correlating with increasing drug exposure times and concentrations, indicating increasing levels of beaded aggregates and larger ABs due to apoptosis. Another impedance metric corresponding to an increase in chemotherapeutic drug concentration can include a downward shift in ϕZ_{18 MHz} for both cellular 204 and sub-cellular 202 populations. This can indicate systematic apoptosis-induced alterations in electrophysiology of the cell interior caused by the chemotherapeutic drug. Yet another impedance metric corresponding to an increase in chemotherapeutic drug concentration can include a drop in mean ϕZ, indicating a decrease in interior cellular conductivity (σint), e.g., related to ionic efflux.

Certain similarities in the respective trends of the ϕZ_{18 MHz} distributions of the cellular 204 and sub-cellular 202 populations indicate that this impedance phase metric can be used to help infer phenotypic similarity between the two populations 202 & 204 due to apoptosis. Furthermore, the sharper drops observed for the ϕZ_{18 MHz} distributions of the sub-cellular populations 202 suggest their greater sensitivity to the onset of apoptosis. Since the sub-cellular gate of the impedance data includes various sub-populations of the ABs, such as smaller micro-vesicles, beaded aggregates of ABs and larger ABs generated by cell disassembly, the culture supernatant media comprising the size fraction of ABs (<5 μm) can be used to help stratify their phenotypes.

FIG. 2C and FIG. 2D depict examples of three dimensional (3D) density plots of impedance metrics for cancer cell samples untreated and exposed to a chemotherapeutic drug, respectively. In an example, a single frequency of 10 MHz can be used for the impedance phase (e.g., ϕZ_{10 MHz}) and electrical size

$$( e . g . {,}^{3} \sqrt{} ( {\vert Z \vert}_{0.5 MHz} ) )$$

measurements to optimize the signal-to-noise ratio for single-particle detection. Here, certain differences in interior electrophysiology can be distinguished, while permitting size normalization, e.g., by co-flowing 5 μm polystyrene reference beads with the cancer cells. As depicted in FIG. 2D, ABs can be stratified based on their size (d) and impedance phase (ϕZ_{10 MHz}) as spherical micro-vesicles 206 (d<2.5 μm; ϕZ_{10 MHz}<0.4, prolate ABs 208 (d˜3-8 μm; ϕZ_{10 MHz}<0.4) or oblate ABs 210 (d˜3-8 μm; ϕZ_{10 MHz}>0.4). Comparison of FIG. 2C and FIG. 2D shows that oblate 210 and prolate 208 ABs rise sharply when subject to drug-induced apoptosis, while spheres 206 increase relatively less with drug treatment.

FIG. 2E and FIG. 2F each depict plots showing impedance metrics for various ABs for treated and untreated cancer cell cultures. In particular, FIG. 2E and FIG. 2F depict normalized events for oblate 210, prolate 208, and spherical 206 ABs versus size and phase, respectively. As shown in FIG. 2E, size distributions do not substantially change for oblate 210 prolate 208 or spherical ABs 206 upon treatment with the chemotherapeutic drug. As shown in FIG. 2F, the ϕZ_{10 MHz} distributions are relatively unchanged after drug treatment for oblate 210 ABs, but shift to higher values for both prolates 208 and spheres 206 after treatment with the chemotherapeutic drug. Such a difference indicates that these increased-phase prolates 208 and spheres 206 correspond with cancer cell-derived membrane-bound vesicles.

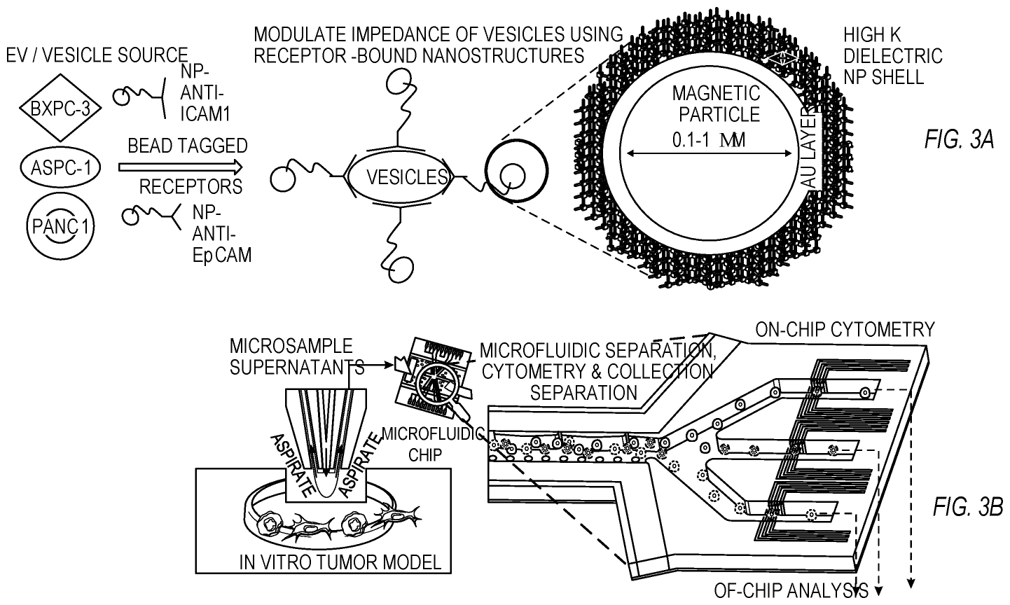

FIG. 3A is a schematic showing modification of a vesicle using receptor-bound nanostructures to help distinguish impedance metrics during impedance cytometry. In an example, the ABs can include modified vesicles include synthetic nanostructures bound with surface proteins. Such synthetic nanostructures, (e.g., polystyrene beads, gold nanoparticles, quantum dots, single-walled carbon nanotubes, etc.) can be bound to a cell-surface receptor, such as an antibody, to recognize specific cell types. For example, the synthetic nanostructures can be magnetic particles having a diameter within a range of about 0.1 μm to about 1 μm. When these nanostructures are bound to a cell, they can interfere with the normal electrical transport of ions across the cell membrane, leading to an altered impedance measurement. The altered ABs can thus be used to distinguish between different cell types or states, e.g., by characteristically altering an impedance characteristic of the vesicle based on protein type and expression level. Micro-vesicles from different cell sources (cancer or non-cancer cell types) can be distinguished by expressed surface markers (e.g., containing epithelial cell adhesion molecule (EpCAM) or intercellular adhesion molecule (ICAM) expression) by binding with receptor-bound nanostructures of different impedances for cytometry and separation of vesicle subpopulations.

FIG. 3B depicts a microfluidic device micro-sampling and microfluidic separation of secreted bodies. In an example, secreted bodies (e.g., floating cells, ABs, and micro-vesicles can be separated from one another via an on-chip microfluidic separation cytometry device. The device can be configured to sort and separate secreted bodies according to their size, charge, and/or other characteristics. The separated secreted bodies can then be collected, analyzed, and used for downstream applications such as off-chip transplantation into cancer models. For example, the separated secreted bodies can be used for in vivo studies of cancer-related signaling pathways, for drug screening, and for cancer immunotherapy.

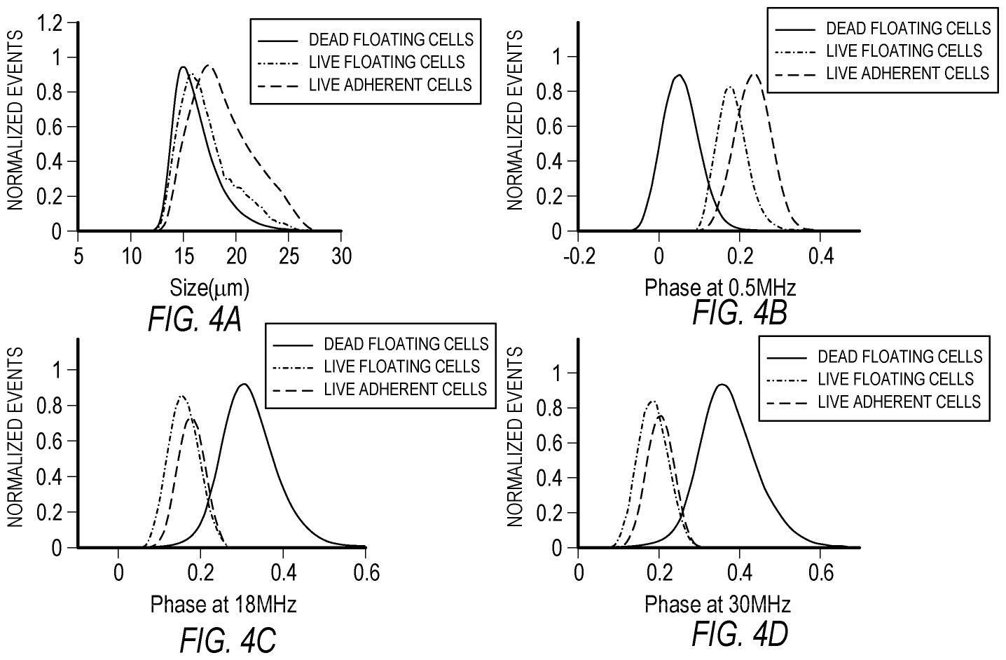

FIG. 4A, FIG. 4B, FIG. 4C, FIG. 4D, are plots of impedance metrics of live floating cancer cells, secreted from chemotherapeutic drug-treated cancer cell cultures, as compared with various phenotypes of the drug-resistant subpopulation of live adherent cells lifted a cancer cell culture. As depicted, there exist significant, detectable similarities between the live floating cancer cells and the live adherent, lifted cells. For example, despite a size of floating cells (live and dead) can be significantly smaller than that of drug-resistant adhered cells (as depicted in FIG. 4A), varying impedance phase metrics (depicted in FIG. 4B, FIG. 4C, and FIG. 4D) show that live floating cancer cells and live adherent, lifted cells exhibit significantly similar phase impedance metrics.

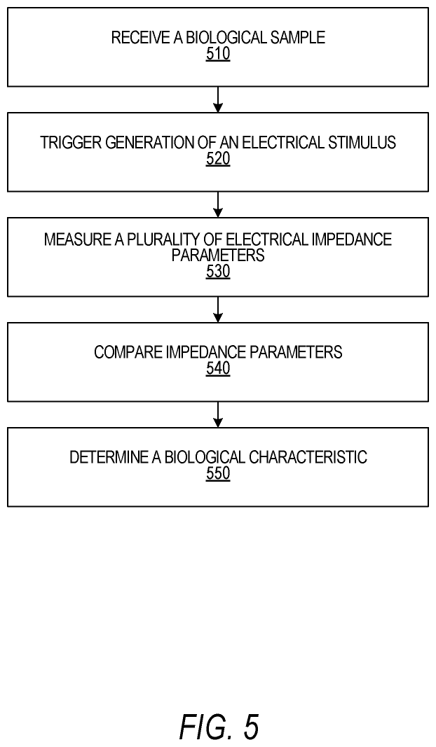

FIG. 5 is a flowchart that describes a machine-implemented method for predicting drug response and toxicity.

In an example, at 510, the machine-implemented method can include receiving a supernatant biological sample of secreted bodies in a flow cell structure that can be obtained from in vitro mammalian tumor tissue concurrent with treatment with a specified chemotherapeutic agent. For example, the received in vitro mammalian tumor cells can correspond to a specified patient-derived tumor. The machine-implemented method can include propagating the in vivo mammalian tumor cells to establish the in vitro mammalian tumor cells used to obtain the biological sample of secreted bodies. In an example, receiving a biological sample in a flow cell structure can include flowing the biological sample at a throughput between about 200 particles per second and about 500 particles per second. In an example, the received biological sample can include synthetic nanostructures that configured to specifically bind with surface proteins on apoptotic body (AB) vesicles to characteristically alter the impedance of the vesicles based on protein type and expression level.

At 520, the machine-implemented method can include triggering generation of an alternating current (AC) electrical stimulus to a set of electrode structures that can be electrically coupled with the flow cell structure for impedance measurement of flowing secreted bodies at a substantially single-particle sensitivity.

In an example, at 530, the machine-implemented method can include measuring, in response to the electrical stimulus, a plurality of electrical impedance magnitude and phase parameters of the biological sample within the flow cell structure, corresponding to a specified range of frequencies. For example, the specified range of frequencies can comprise frequencies within a range of about 500 kilohertz to about 50 megahertz.

At 540, the machine-implemented method can include comparing the measured electrical impedance parameters of the biological sample with respective electrical impedance parameters corresponding to at least one of model live floating cells, model apoptotic cells, model apoptotic bodies, or micro-vesicles corresponding with apoptotic cells. For example, such comparing can include comparing a measured impedance phase versus size distribution of the biological sample with impedance phase versus size distribution of the model apoptotic cells and live floating cells.

At 550, the machine-implemented method can include determining, based on the compared parameters, a biological characteristic corresponding to a chemotherapeutic drug sensitivity of the in vitro mammalian tumor cells in the biological sample. For example, such determining can include determining a biological characteristic corresponding with at least one of live, apoptotic or necrotic states of the in vitro mammalian tumor cells in the biological sample. Also, determining the biological characteristic can include determining a presence of apoptotic bodies (ABs) and micro-vesicles in the biological sample.

The machine-implemented method can also include distinguishing subpopulations of vesicles corresponding to the ABs based on the measured impedance phase and size distribution of the biological sample. Here, the machine-implemented method can include determining, based on the distinguished subpopulations, a drug resistance of the in vitro mammalian tumor cells to the specified chemotherapeutic agent. For example, a specific subpopulation of vesicles from the biological sample can be isolated, e.g., based on their detected impedance characteristics. The machine-implemented method can include comparing the distinguished subpopulations of vesicles corresponding to the ABs with a tumor microenvironment (TME) model. The machine-implemented method can include determining a preliminary indicator of efficacy of the chemotherapeutic agent based on imaging of in vivo mammalian tumor cells. The machine-implemented method can also include establishing or adjusting a drug treatment plan based on the determined at least one distinguishable biophysical feature of the biological sample.

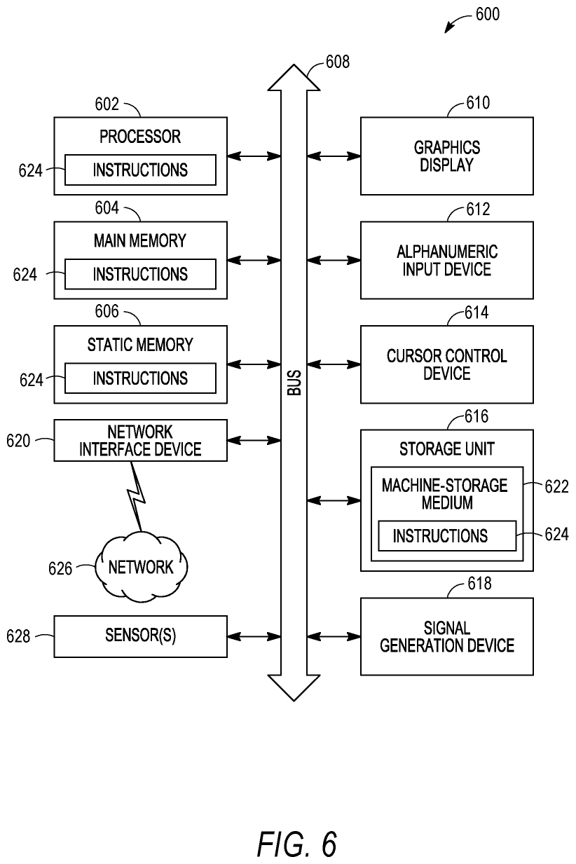

FIG. 6 is a block diagram illustrating components of a machine 600, according to some example embodiments, able to read instructions 624 from a machine-storage medium 622 (e.g., a non-transitory machine-storage medium, a machine-storage medium, a computer-storage medium, or any suitable combination thereof) and perform any one or more of the methodologies discussed herein, in whole or in part. Specifically, FIG. 6 shows the machine 600 in the example form of a computer system (e.g., a computer) within which the instructions 624 (e.g., software, a program, an application, an applet, an app, or other executable code) for causing the machine 600 to perform any one or more of the methodologies discussed herein can be executed, in whole or in part. For example, the instructions 624 can be processor executable instructions that, when executed by a processor of the machine 600, cause the machine 600 to perform the operations outlined above.

In various embodiments, the machine 600 operates as a standalone device or can be communicatively coupled (e.g., networked) to other machines. In a networked deployment, the machine 600 can operate in the capacity of a server machine or a client machine in a server-client network environment, or as a peer machine in a distributed (e.g., peer-to-peer) network environment. The machine 600 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 624, sequentially or otherwise, that specify actions to be taken by that machine. Further, while only a single machine is illustrated, the term “machine” shall also be taken to include any collection of machines that individually or jointly execute the instructions 624 to perform all or part of any one or more of the methodologies discussed herein.

The machine 600 includes a processor 602 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 604, and a static memory 606, which are configured to communicate with each other via a bus 608. The processor 602 can contain microcircuits that are configurable, temporarily or permanently, by some or all of the instructions 624 such that the processor 602 is configurable to perform any one or more of the methodologies described herein, in whole or in part. For example, a set of one or more microcircuits of the processor 602 can be configurable to execute one or more modules (e.g., software modules) described herein.

The machine 600 can further include a graphics display 610 (e.g., a plasma display panel (PDP), a light emitting diode (LED) display, a liquid crystal display (LCD), a projector, a cathode ray tube (CRT), or any other display capable of displaying graphics or video). The machine 600 can also include an alphanumeric input device 612 (e.g., a keyboard or keypad), a cursor control device 614 (e.g., a mouse, a touchpad, a trackball, a joystick, a motion sensor, an eye tracking device, or other pointing instrument), a storage unit 616, an audio generation device 618 (e.g., a sound card, an amplifier, a speaker, a headphone jack, any suitable combination thereof, or any other suitable signal generation device), and a network interface device 620.

The storage unit 616 includes the machine-storage medium 622 (e.g., a tangible and non-transitory machine-storage medium) on which are stored the instructions 624, embodying any one or more of the methodologies or functions described herein. The instructions 624 can also reside, completely or at least partially, within the main memory 604, within the processor 602 (e.g., within the processor's cache memory), or both, before or during execution thereof by the machine 600. Accordingly, the main memory 604 and the processor 602 can be considered machine-storage media (e.g., tangible and non-transitory machine-storage media). The instructions 624 can be transmitted or received over the network 626 via the network interface device 620. For example, the network interface device 620 can communicate the instructions 624 using any one or more transfer protocols (e.g., Hypertext Transfer Protocol (HTTP)).

In some example embodiments, the machine 600 can be a portable computing device, such as a smart phone or tablet computer, and have one or more additional input components (e.g., sensors 628 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 604, 606, and/or memory of the processor(s) 602) and/or storage unit 616 can store one or more sets of instructions and data structures (e.g., software) 624 embodying or utilized by any one or more of the methodologies or functions described herein. These instructions, when executed by processor(s) 602 cause various operations to implement the disclosed embodiments.

As used herein, the terms “machine-storage medium,” “device-storage medium,” “computer-storage medium” (referred to collectively as “machine-storage medium 622”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 622 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 622 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The following, non-limiting examples, detail certain aspects of the present subject matter to solve the challenges and provide the benefits discussed herein, among others.

Aspect 1 is a machine-implemented method for predicting drug response and toxicity using in vitro mammalian tumor cells, the machine-implemented method comprising: receiving a supernatant biological sample of secreted bodies in a flow cell structure that are obtained from in vitro mammalian tumor tissue concurrent with treatment with a specified chemotherapeutic agent; triggering generation of an alternating current (AC) electrical stimulus to a set of electrode structures that are electrically coupled with the flow cell structure for impedance measurement of flowing secreted bodies at a substantially single-particle sensitivity; measuring, in response to the electrical stimulus, a plurality of electrical impedance magnitude and phase parameters of the biological sample within the flow cell structure, corresponding to a specified range of frequencies; comparing the measured electrical impedance parameters of the biological sample with respective electrical impedance parameters corresponding to at least one of model live floating cells, model apoptotic cells, model apoptotic bodies, or micro-vesicles corresponding with apoptotic cells; and determining, based on the compared parameters, a biological characteristic corresponding to a chemotherapeutic drug sensitivity of the in vitro mammalian tumor cells in the biological sample.

In Aspect 2, the subject matter of Aspect 1 includes, wherein the received in vitro mammalian tumor cells corresponds to a specified patient-derived tumor.

In Aspect 3, the subject matter of any one of Aspects 1-2 includes, wherein the determining, based on the compared parameters, includes determining a biological characteristic corresponding with at least one of live, apoptotic, or necrotic states of the in vitro mammalian tumor cells in the biological sample.

In Aspect 4, the subject matter of any one of Aspects 1-3 includes, wherein determining the biological characteristic includes determining a presence of apoptotic bodies (ABs) and micro-vesicles in the biological sample.

In Aspect 5, the subject matter of Aspect 4 includes, wherein comparing the measured electrical impedance parameters includes comparing a measured impedance phase versus size distribution of the biological sample with impedance phase versus size distribution of the model apoptotic cells and live floating cells.

In Aspect 6, the subject matter of Aspect 5 includes, receiving synthetic nanostructures configured to specifically bind with surface proteins of a cellular vesicle to characteristically alter an impedance characteristic of the vesicle based on a protein type and expression level of the vesicle; distinguishing at least one subpopulation of nanostructure-altered cellular vesicles corresponding to the ABs based on the measured impedance phase and size distribution of the biological sample; isolating an individual subpopulation of the nanostructure-altered cellular vesicles from the biological sample based on at least one impedance characteristic; and determining, based on the distinguished at least one subpopulation, a drug resistance of the in vitro mammalian tumor cells to the specified chemotherapeutic agent.

In Aspect 7, the subject matter of any one of Aspects 5-6 includes, facilitating binding synthetic nanostructures with surface proteins of a cellular vesicle to characteristically alter the measured impedance based on protein type and expression level of the vesicle.

In Aspect 8, the subject matter of any one of Aspects 6-7 includes, comparing the distinguished subpopulations of vesicles corresponding to the ABs with a tumor microenvironment (TME) model.

In Aspect 9, the subject matter of any one of Aspects 1-8 includes, establishing or adjusting a drug treatment plan based on the determined at least one distinguishable biophysical feature of the biological sample.

In Aspect 10, the subject matter of any one of Aspects 1-9 includes, wherein the specified range of frequencies comprises frequencies within a range of 500 kilohertz to 50 megahertz.

In Aspect 11, the subject matter of any one of Aspects 1-10 includes, wherein receiving a biological sample in a flow cell structure includes flowing the biological sample at a throughput within a range of 200-500 particles per second.

In Aspect 12, the subject matter of any one of Aspects 1-11 includes, determining a preliminary indicator of efficacy of the chemotherapeutic agent based on imaging of in vivo mammalian tumor cells.

In Aspect 13, the subject matter of Aspect 12 includes, propagating the in vivo mammalian tumor cells to establish the in vitro mammalian tumor cells used to obtain the biological sample of secreted bodies.

Aspect 14 is at least one non-transitory machine-readable medium including instructions for predicting drug response and toxicity using in vitro mammalian tumor cells, which when executed by a processor, cause the processor to: identify a supernatant biological sample of secreted bodies in a flow cell structure that are obtained from in vitro mammalian tumor tissue concurrent with treatment with a specified chemotherapeutic agent; trigger generation of an alternating current (AC) electrical stimulus to a set of electrode structures that are electrically coupled with the flow cell structure for impedance measurement of flowing secreted bodies at a substantially single-particle sensitivity; measure, in response to the electrical stimulus, a plurality of electrical impedance magnitude and phase parameters of the biological sample within the flow cell structure, corresponding to a specified range of frequencies; compare the measured electrical impedance parameters of the biological sample with respective electrical impedance parameters corresponding to at least one of model live floating cells, model apoptotic cells, model apoptotic bodies, or micro-vesicles corresponding with apoptotic cells; and determine, based on the compared parameters, a biological characteristic corresponding to a chemotherapeutic drug sensitivity of the in vitro mammalian tumor cells in the biological sample.

In Aspect 15, the subject matter of Aspect 14 includes, wherein the identified in vitro mammalian tumor cells correspond to a specified patient-derived tumor.

In Aspect 16, the subject matter of any one of Aspects 14-15 includes, instructions which cause the processor to determine a biological characteristic corresponding with at least one of live, apoptotic, or necrotic states of the in vitro mammalian tumor cells in the biological sample.

In Aspect 17, the subject matter of any one of Aspects 14-16 includes, instructions which cause the processor to determine a presence of apoptotic bodies (ABs) and micro-vesicles in the biological sample.

In Aspect 18, the subject matter of Aspect 17 includes, instructions which cause the processor to compare a measured impedance phase versus size distribution of the biological sample with impedance phase versus size distribution of the model apoptotic cells and live floating cells.

In Aspect 19, the subject matter of Aspect 18 includes, instructions which cause the processor to: receive synthetic nanostructures configured to specifically bind with surface proteins a vesicle to characteristically alter an impedance characteristic of the vesicle based on protein type and expression level; distinguish at least one subpopulation of vesicles corresponding to the ABs based on the measured impedance phase and size distribution of the biological sample; isolate an individual subpopulation of vesicles from the biological sample based on at least one impedance characteristic; and determine, based on the distinguished at least one subpopulation, a drug resistance of the in vitro mammalian tumor cells to the specified chemotherapeutic agent.

In Aspect 20, the subject matter of Aspect 19 includes, instructions which cause the processor to compare the distinguished subpopulations of vesicles corresponding to the ABs with a tumor microenvironment (TME) model.

In Aspect 21, the subject matter of any one of Aspects 14-20 includes, instructions which cause the processor to establish or adjust a drug treatment plan based on the determined at least one distinguishable biophysical feature of the biological sample.

In Aspect 22, the subject matter of any one of Aspects 14-21 includes, wherein the specified range of frequencies comprises frequencies within a range of 500 kilohertz to 50 megahertz.

In Aspect 23, the subject matter of any one of Aspects 14-22 includes, wherein receiving a biological sample in a flow cell structure includes flowing the biological sample at a throughput within a range of 200-500 particles per second.

In Aspect 24, the subject matter of any one of Aspects 14-23 includes, determining a preliminary indicator of efficacy of the chemotherapeutic agent based on imaging of in vivo mammalian tumor cells.

In Aspect 25, the subject matter of Aspect 24 includes, propagating the in vivo mammalian tumor cells to establish the in vitro mammalian tumor cells used to obtain the biological sample of secreted bodies.

Aspect 26 is a microfluidic system for predicting drug response and toxicity using in vitro mammalian tumor cells, the microfluidic system comprising: a main channel defining an inlet and an outlet, the main channel configured to receive a supernatant biological sample of secreted bodies obtained from in vitro mammalian tumor tissue concurrent with treatment with a specified chemotherapeutic agent, at the inlet; a set of electrode structures that are coupled with the main channel for impedance measurement of flowing secreted bodies at a substantially single-particle sensitivity; a waveform generator configured to deliver an alternating current (AC) electrical stimulus to a set of electrode structures; and a processor configured to: measure, via the electrodes in response to the electrical stimulus, a plurality of electrical impedance magnitude and phase parameters of the biological sample within the flow cell structure, corresponding to a specified range of frequencies; compare the measured electrical impedance parameters of the biological sample with respective electrical impedance parameters corresponding to at least one of model live floating cells, model apoptotic cells, model apoptotic bodies, or micro-vesicles corresponding with apoptotic cells; and determine, based on the compared parameters, a biological characteristic corresponding to a chemotherapeutic drug sensitivity of the in vitro mammalian tumor cells in the biological sample.

In Aspect 27, the subject matter of Aspect 26 includes, wherein the in vitro mammalian tumor cells, received in the main channel, correspond to a specified patient-derived tumor.

In Aspect 28, the subject matter of any one of Aspects 26-27 includes, wherein the processor is configured to determine a biological characteristic corresponding with at least one of live, apoptotic, or necrotic states of the in vitro mammalian tumor cells in the biological sample.

In Aspect 29, the subject matter of any one of Aspects 26-28 includes, wherein the processor is configured to determine a presence of apoptotic bodies (ABs) and micro-vesicles in the biological sample.

In Aspect 30, the subject matter of Aspect 29 includes, wherein the processor is configured to compare a measured impedance phase versus size distribution of the biological sample with impedance phase versus size distribution of the model apoptotic cells and live floating cells.

In Aspect 31, the subject matter of Aspect 30 includes, wherein the processor is configured to: distinguish subpopulations of modified vesicles corresponding to the ABs based on the measured impedance phase and size distribution of the biological sample, the modified vesicles including synthetic nanostructures bound with surface proteins a vesicle to characteristically alter an impedance characteristic of the vesicle based on protein type and expression level; and determine, based on the distinguished subpopulations, a drug resistance of the in vitro mammalian tumor cells to the specified chemotherapeutic agent.

In Aspect 32, the subject matter of Aspect 31 includes, wherein the processor is configured to compare the distinguished subpopulations of vesicles corresponding to the ABs with a tumor microenvironment (TME) model.

In Aspect 33, the subject matter of any one of Aspects 26-32 includes, wherein the processor is configured to generate instructions for establishing or adjusting a drug treatment plan based on the determined at least one distinguishable biophysical feature of the biological sample.

In Aspect 34, the subject matter of any one of Aspects 26-33 includes, wherein the specified range of frequencies comprises frequencies within a range of 500 kilohertz to 50 megahertz.

In Aspect 35, the subject matter of any one of Aspects 26-34 includes, at least one focusing flow channel feeding the main channel to establish or adjust a flow rate of the biological sample at a throughput within a range of 200-500 particles per second.

In Aspect 36, the subject matter of any one of Aspects 26-35 includes, wherein the processor is configured to determine a preliminary indicator of efficacy of the chemotherapeutic agent based on imaging of in vivo mammalian tumor cells.

Aspect 37 is at least one machine-readable medium including instructions that, when executed by processing circuitry, cause the processing circuitry to perform operations to implement of any of Aspects 1-36.

Aspect 38 is an apparatus comprising means to implement of any one of Aspects 1-36.

Aspect 39 is a system to implement of any one of Aspects 1-36.

Aspect 40 is a method to implement of any one of Aspects 1-36.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to help allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A machine-implemented method for predicting drug response and toxicity using in vitro mammalian tumor cells, the machine-implemented method comprising:
receiving a supernatant biological sample of secreted bodies in a flow cell structure that are obtained from in vitro mammalian tumor cells concurrent with treatment with a specified chemotherapeutic agent;
triggering generation of an alternating current (AC) electrical stimulus to a set of electrode structures that are electrically coupled with the flow cell structure for impedance measurement of flowing secreted bodies at a substantially single-particle sensitivity;
measuring, in response to the electrical stimulus, a plurality of electrical impedance parameters of the biological sample within the flow cell structure, corresponding to a specified range of frequencies;
comparing the measured electrical impedance parameters of the biological sample with respective electrical impedance parameters corresponding to model apoptotic cells; and
determining, based on the compared parameters, a biological characteristic corresponding to a chemotherapeutic drug sensitivity of the in vitro mammalian tumor cells in the biological sample.

2. The machine-implemented method of claim 1, wherein the received in vitro mammalian tumor cells corresponds to a specified patient-derived tumor.

3. The machine-implemented method of claim 1, wherein the determining, based on the compared parameters, includes determining a biological characteristic corresponding with at least one of live, apoptotic, or necrotic states of the in vitro mammalian tumor cells in the biological sample.

4. The machine-implemented method of claim 1, wherein determining the biological characteristic includes determining a presence of apoptotic bodies (ABs) in the biological sample.

5. The machine-implemented method of claim 4, wherein comparing the measured electrical impedance parameters includes comparing a measured impedance phase versus size distribution of the biological sample with impedance phase versus size distribution of the model apoptotic cells.

6. The machine-implemented method of claim 5, comprising:
receiving synthetic nanostructures configured to specifically bind with surface proteins of a cellular vesicle to characteristically alter an impedance characteristic of the vesicle based on a protein type and expression level of the vesicle;
distinguishing at least one subpopulation of nanostructure-altered cellular vesicles corresponding to the ABs based on the measured impedance phase and size distribution of the biological sample;
isolating an individual subpopulation of the nanostructure-altered cellular vesicles from the biological sample based on at least one impedance characteristic; and
determining, based on the distinguished at least one subpopulation, a drug resistance of the in vitro mammalian tumor cells to the specified chemotherapeutic agent.

7. The machine-implemented method of claim 5, comprising:
facilitating binding synthetic nanostructures with surface proteins of a cellular vesicle to characteristically alter the measured impedance based on protein type and expression level of the vesicle.

8-13. (canceled)

14. At least one non-transitory machine-readable medium including instructions for predicting drug response and toxicity using in vitro mammalian tumor cells, which when executed by a processor, cause the processor to:
identify a supernatant biological sample of secreted bodies in a flow cell structure that are obtained from in vitro mammalian tumor tissue concurrent with treatment with a specified chemotherapeutic agent;
trigger generation of an alternating current (AC) electrical stimulus to a set of electrode structures that are electrically coupled with the flow cell structure for impedance measurement of flowing secreted bodies at a substantially single-particle sensitivity;
measure, in response to the electrical stimulus, a plurality of electrical impedance parameters of the biological sample within the flow cell structure, corresponding to a specified range of frequencies;
compare the measured electrical impedance parameters of the biological sample with respective electrical impedance parameters corresponding to model apoptotic cells; and
determine, based on the compared parameters, a biological characteristic corresponding to a chemotherapeutic drug sensitivity of the in vitro mammalian tumor cells in the biological sample.

15. The at least one machine-readable medium of claim 14, wherein the identified in vitro mammalian tumor cells correspond to a specified patient-derived tumor.

16. The at least one machine-readable medium of claim 14, including instructions which cause the processor to determine a biological characteristic corresponding with at least one of live, apoptotic, or necrotic states of the in vitro mammalian tumor cells in the biological sample.

17. The at least one machine-readable medium of claim 14, including instructions which cause the processor to determine a presence of apoptotic bodies (ABs) in the biological sample.

18. The at least one machine-readable medium of claim 17, including instructions which cause the processor to compare a measured impedance phase versus size distribution of the biological sample with impedance phase versus size distribution of the model apoptotic cells.

19. The at least one machine-readable medium of claim 18, including instructions which cause the processor to:
characterize, based on electrical impedance parameters, synthetic nanostructures configured to specifically bind with surface proteins of a cellular vesicle to characteristically alter an impedance characteristic of the vesicle based on protein type and expression level;
distinguish at least one subpopulation of vesicles corresponding to the ABs based on the measured impedance phase and size distribution of the biological sample;
isolate an individual subpopulation of vesicles from the biological sample based on at least one impedance characteristic; and
determine, based on the distinguished at least one subpopulation, a drug resistance of the in vitro mammalian tumor cells to the specified chemotherapeutic agent.

20-25. (canceled)

26. A microfluidic system for predicting drug response and toxicity using in vitro mammalian tumor cells, the microfluidic system comprising:
a main channel defining an inlet and an outlet, the main channel configured to receive a supernatant biological sample of secreted bodies obtained from in vitro mammalian tumor tissue concurrent with treatment with a specified chemotherapeutic agent, at the inlet;
a set of electrode structures that are coupled with the main channel for impedance measurement of flowing secreted bodies at a substantially single-particle sensitivity;
a waveform generator configured to deliver an alternating current (AC) electrical stimulus to a set of electrode structures; and
a processor configured to:
measure, via the electrodes in response to the electrical stimulus, a plurality of electrical impedance parameters of the biological sample within the flow cell structure, corresponding to a specified range of frequencies;
compare the measured electrical impedance parameters of the biological sample with respective electrical impedance parameters corresponding to model apoptotic cell; and
determine, based on the compared parameters, a biological characteristic corresponding to a chemotherapeutic drug sensitivity of the in vitro mammalian tumor cells in the biological sample.

27. The microfluidic system of claim 26, wherein the in vitro mammalian tumor cells, received in the main channel, correspond to a specified patient-derived tumor.

28. The microfluidic system of claim 26, wherein the processor is configured to determine a biological characteristic corresponding with at least one of live, apoptotic, or necrotic states of the in vitro mammalian tumor cells in the biological sample.

29. The microfluidic system of claim 26, wherein the processor is configured to determine a presence of apoptotic bodies (ABs) and micro-vesicles in the biological sample.

30. The microfluidic system of claim 29, wherein the processor is configured to compare a measured impedance phase versus size distribution of the biological sample with impedance phase versus size distribution of the model apoptotic cells and live floating cells.

31. The microfluidic system of claim 30, wherein the processor is configured to:
characterize, based on electrical impedance parameters, synthetic nanostructures configured to specifically bind with surface proteins of a cellular vesicle to characteristically alter an impedance characteristic of the vesicle based on protein type and expression level;
distinguish subpopulations of modified vesicles corresponding to the ABs based on the measured impedance phase and size distribution of the biological sample, the modified vesicles including synthetic nanostructures bound with surface proteins a vesicle to characteristically alter an impedance characteristic of the vesicle based on protein type and expression level; and
determine, based on the distinguished subpopulations, a drug resistance of the in vitro mammalian tumor cells to the specified chemotherapeutic agent.

32-36. (canceled)
